(19) United States
(12) Patent Application Publication
(10) Pub. No.: US 2024/0075470 A1
(43) Pub. Date: Mar. 7, 2024

(54) MICROCHANNEL DEVICE

(71) Applicant: SHIMADZU CORPORATION, Kyoto (JP)

(72) Inventor: Ippei TAKEUCHI, Kyoto-shi (JP)

(21) Appl. No.: 18/237,530

(22) Filed: Aug. 24, 2023

(30) Foreign Application Priority Data
Sep. 6, 2022 (JP) 2022-141464

(51) Int. Cl. B01L 3/00

(52) U.S. Cl. CPC B01L 3/502715; B01L 2300/0816; B01L 2300/0861

(57) ABSTRACT

A microchannel device includes an opening that receives a test solution, a main channel that communicates with the opening, and a collection portion provided at an outlet-side end of the main channel. The collection portion includes a pool that stores the test solution, a connection channel that connects the pool with the outlet-side end, and a protrusion that is arranged in the connection channel to generate air bubbles between the protrusion and an inner wall of the connection channel to close the connection channel, upon receiving the test solution discharged from the main channel.

### CROSS REFERENCE TO RELATED APPLICATIONS

This nonprovisional application is based on Japanese Patent Application No. 2022-141464 filed on Sep. 6, 2022 with the Japan Patent Office, the entire content of which is hereby incorporated by reference.

### BACKGROUND OF THE INVENTION

### Field of the Invention

The present disclosure relates to a microchannel device in a form of a plate used for a test in which a test solution containing a sample and an agent act on each other.

### Description of the Background Art

In order to test sensitivity or the like of bacteria to an antimicrobial, a test method using a microchannel device has been known. For example, in Japanese Patent Laying-Open No. 2017-67620, in a microchannel device including an introduction port and a discharge port that communicate with the outside and a channel through which a test solution supplied from the introduction port flows toward the discharge port, by injecting air into the channel from the introduction port, the test solution introduced previously is pressed into small channels. The channel is provided with a reaction portion where the test solution supplied from the introduction port is stored and an agent arranged in the reaction portion acts on bacteria.

Japanese Patent Laying-Open No. 2022-044563 discloses a test apparatus in which a test solution is injected into a plurality of microchannels to inject the test solution into the microchannels and, thereafter, air pressure is applied to collect the test solution in a main channel communicating with the plurality of microchannels into a collection portion.

### SUMMARY OF THE INVENTION

According to the test apparatus disclosed in Japanese Patent Laying-Open No. 2022-044563, by discharging the test solution that remains in the main channel to the collection portion, the plurality of microchannels are made independent from each other, so that it is possible to suppress a flow of the test solution produced in the channel. In other words, to make the plurality of microchannels independent from each other, there is a demand for the test solution to be surely discharged from the main channel to the collection portion, and for the test solution collected in the collection portion to be retained in the collection portion.

The present disclosure was made to solve such a problem, and it is an object of the present disclosure to cause a test solution discharged to a collection portion to be retained in the collection portion.

A microchannel device of the present disclosure is a microchannel device in a form of a plate used for a test in which a test solution containing a sample and an agent act on each other. The microchannel device includes an opening that receives the test solution; a main channel that communicates with the opening; a plurality of microchannels that each communicate with the main channel; and a collection portion that is provided in the main channel at an outlet-side end, the outlet-side end being located opposite to an inlet-side end communicating with the opening, the collection portion partly collecting the test solution. The collection portion includes a pool that stores the test solution discharged from the main channel, a connection channel that connects the pool with the outlet-side end, and a protrusion that is arranged in the connection channel to generate air bubbles between the protrusion and an inner wall of the connection channel to close the connection channel, upon receiving the test solution discharged from the main channel.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a diagram showing an exemplary overall construction of a test apparatus;

FIG. 2 is a block diagram for illustrating control of the test apparatus;

FIG. 3 is a plan view of a microchannel device;

FIG. 4 is a flowchart for illustrating an injection method of the test apparatus;

FIG. 5 is a diagram for illustrating a flow of a test solution when the test solution is injected by the test apparatus;

FIG. 6 is a perspective view of a collection portion;

FIG. 7 is a cross-sectional view of the collection portion;

FIG. 8 is a plan view of the collection portion;

FIG. 9 is a diagram schematically showing states of the collection portion before and after test solution is discharged;

FIG. 10 is an image showing a state after the test solution is discharged to the collection portion;

FIG. 11 is an image showing a state after a test solution is discharged to a collection portion in a comparison example; and

FIG. 12 is a cross-sectional view of a collection portion according to a modification.

### DESCRIPTION OF THE PREFERRED EMBODIMENT

An embodiment will be described below in detail with reference to the drawings. The same or corresponding elements in the drawings have the same reference characters allotted and description thereof will not be repeated.

[Apparatus Construction of Test Apparatus]

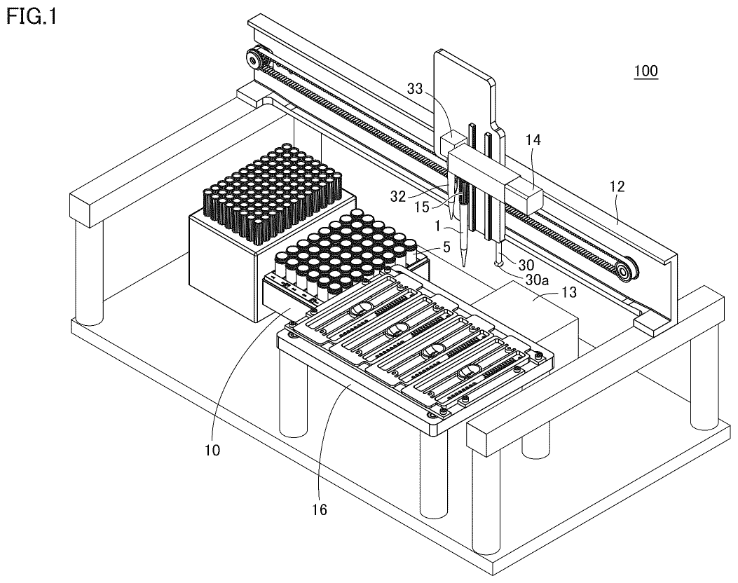

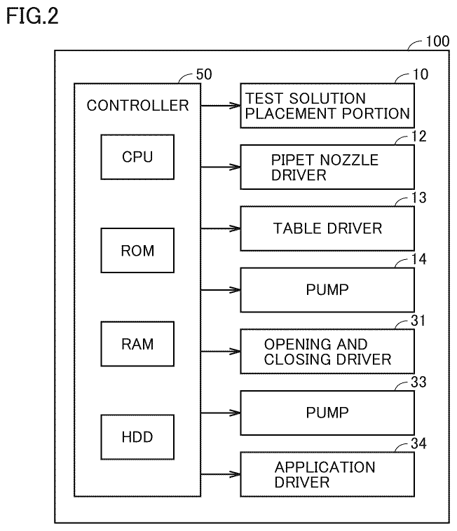

Referring to FIG. 1 and FIG. 2, a test apparatus 100 for pouring a test solution into a microchannel device will be described. FIG. 1 is a diagram showing an exemplary overall construction of the test apparatus. FIG. 2 is a block diagram for illustrating control of the test apparatus. The test apparatus according to the present embodiment is an apparatus for measurement of a test solution containing a sample by injecting the test solution into a microchannel in the microchannel device, and an example in which the test solution is injected into a microchannel for measuring sensitivity of bacteria to an antimicrobial (agent) is described below by way of example. The test solution contains a sample. The sample may be bacteria (pathogenic bacteria in a specific example). In the specific example, the test solution may be a suspension of bacteria. Naturally, for the test solution, so long as a test solution is injected into a microchannel in the microchannel device by the test apparatus, limitation to the test solution described above is not intended.

Referring to FIG. 1 and FIG. 2, test apparatus 100 includes a test solution placement portion 10, a pipet nozzle driver 12, a table driver 13, a pump 14, a pipet nozzle 15, a table 16, an opening and closing unit 30, an opening and closing driver 31, an applicator 32, a pump 33, an application driver 34, and a controller 50.

Test solution placement portion 10 is a rack where a plurality of test solution containers 5 each containing a test solution can be arranged. In test solution placement portion 10, a plurality of test solution containers 5 can be set on test apparatus 100, in a unit of a rack.

Pipet nozzle 15 having a removable pipet chip 1 attached thereto suctions or discharges a test solution from test solution container 5 through a tip end of pipet chip 1.

Pipet nozzle driver 12 horizontally moves pipet nozzle 15, pump 14 connected to pipet nozzle 15, opening and closing unit 30, applicator 32, and pump 33 connected to applicator 32. Pipet nozzle driver 12 vertically moves pipet nozzle 15 and pump 14 connected to pipet nozzle 15. Pipet nozzle driver 12 can freely move pipet nozzle 15, for example, by means of a solenoid actuator or a stepping motor.

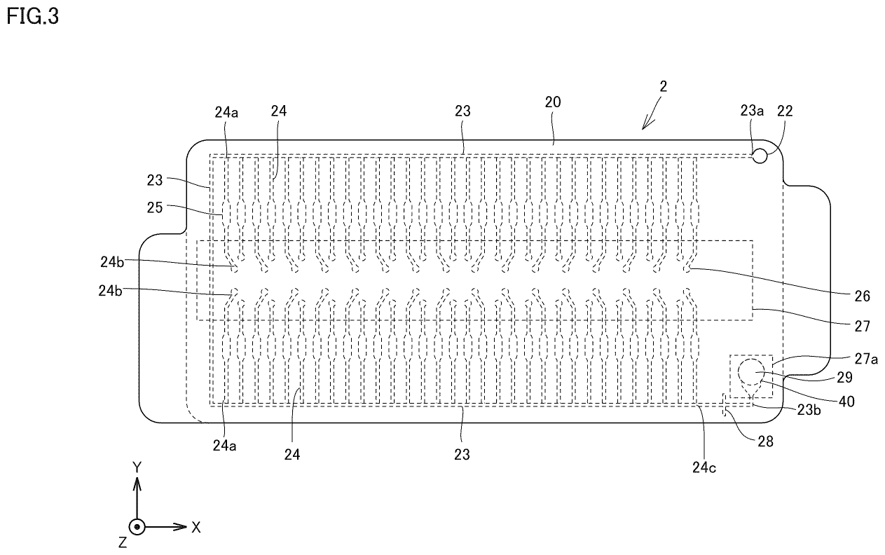

Table 16 is a support member on which a microchannel device 2 (see FIG. 3) is carried Table 16 is in a shape of a flat plate and microchannel device 2 is fixed to an upper surface thereof. Table driver 13 can horizontally move table 16. Table driver 13 can freely move table 16, for example, by means of a solenoid actuator or a stepping motor. Naturally, table driver 13 may vertically move table 16 so that pipet nozzle 15 is not vertically moved. At least pipet nozzle driver 12 and table driver 13 are each a movement mechanism for changing a position of pipet nozzle 15 and a position of microchannel device 2 relative to each other.

Though not shown, pump 14 includes, for example, a syringe, a plunger capable of carrying out reciprocating motion within the syringe, and a drive motor that drives the plunger. Pump 14 can regulate an air pressure in pipet chip 1 by causing the plunger to carry out reciprocating motion while the plunger is connected to pipet nozzle 15 through a pipe to suction the test solution into pipet chip 1 or discharge the test solution in pipet chip 1 to the outside. Pump 14 can deliver air to the outside of pipet chip 1 by moving the plunger further into the syringe with the test solution in pipet chip 1 having been discharged to the outside.

Opening and closing unit 30 is a mechanism that opens or closes an opening 29 (see FIG. 3) formed in microchannel device 2 which will be described later. Opening and closing unit 30 includes an elastic member 30a provided at a tip end of a rod-shaped support portion. Elastic member 30a may be made of a silicone resin, for example. Opening and closing driver 31 drives opening and closing unit 30 to vertically move elastic member 30a. Opening and closing driver 31 vertically moves elastic member 30a that has moved to the position right above opening 29 to press elastic member 30a against or to separate elastic member 30a from a gas permeable membrane 27a that covers opening 29. Thus, opening and closing unit 30 including elastic member 30a opens or closes opening 29. Though FIG. 1 shows the construction in which opening and closing unit 30 and pipet nozzle 15 are provided in the same movement mechanism, opening and closing unit 30 may be provided in a movement mechanism different from the movement mechanism where pipet nozzle 15 is provided and opening and closing driver 31 may move opening and closing unit 30.

Applicator 32 applies a sealing material to an opening or the like formed in microchannel device 2. Applicator 32 is implemented, for example, by a nozzle that discharges the sealing material, such as silicone oil, to the opening or the like, and applies the sealing material to the opening or the like front the nozzle by means of pump 33. The construction of applicator 32 is not limited as such, and a mechanism that applies the sealing material to the opening or the like with a brush or the like may be applicable.

Application driver 34 moves applicator 32 to a position at which the sealing material is to be applied, and drives pump 33. Though FIG. 1 shows the construction in which applicator 32 and pipet nozzle 15 are provided in the same movement mechanism, applicator 32 may be provided in a movement mechanism different from a movement mechanism where pipet nozzle 15 is provided and application driver 34 may move applicator 32.

Controller 50 controls an operation of test apparatus 100. Controller 50 includes a processor such as a central processing unit (CPU) and a memory such as a read only memory (ROM) and a random access memory (RAM). A control program is stored in the memory. The processor controls the operation of test apparatus 100 by execution of the control program. The memory of controller 50 may include a hard disk drive (HDD).

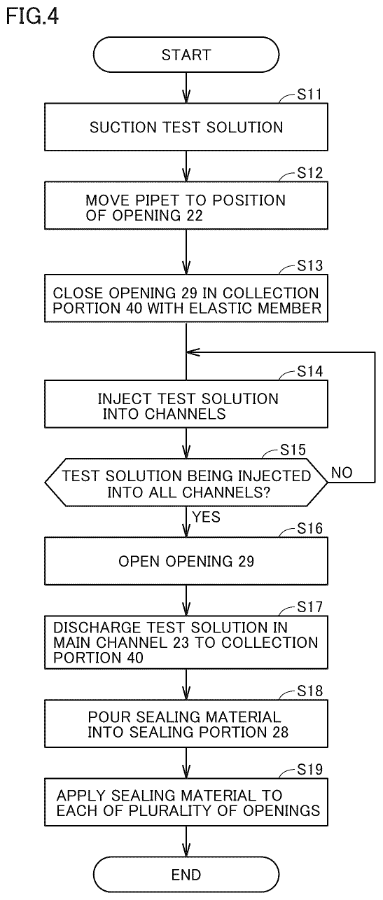

As shown in FIG. 2, controller 50 controls test solution placement portion 10, pipet nozzle driver 12, table driver 13, pump 14, opening and closing driver 31, pump 33, and application driver 34. Controller 50 controls such components to pour a test solution into microchannel device 2 placed on table 16 or to apply a sealing material to the opening or the like formed in microchannel device 2. A specific operation of test apparatus 100 will be described later with reference to FIG. 4.

A computing processor, such as a computer, that a user uses to manage test apparatus 100 may be connected to controller 50. The computing processor receives, for example, inputs for an amount of movement of table 16 and an amount of a test solution to be poured into microchannel device 2

[Construction of Microchannel Device]

FIG. 3 is a plan view of the microchannel device. Microchannel device 2 is placed on table 16 of test apparatus 100. In the description made hereinafter, the surface of microchannel device 2 provided with an opening 22 is assumed as an XY plane, and an axis orthogonal to the XY plane is assumed as a Z axis. Hereinafter, the surface on a side where opening 22 is provided is assumed as an upper surface and a surface that faces the upper surface is assumed as a bottom surface, and a positive direction of the Z axis may be referred to as an upward direction and a negative direction of the Z axis may be referred to as a downward direction.

Microchannel device 2 is placed on table 16 with the bottom surface having no opening 22 used as a placement surface. As shown in FIG. 3, microchannel device 2 includes a plate-shaped member 20 and a channel structure. The channel structure includes opening 22, a main channel 23, microchannels 24, reservoirs 25, openings 26, and a collection portion 40 provided with opening 29.

Opening 22 is connected to an inlet-side end 23a, being one end of main channel 23, and communicates with main channel 23. The test solution is injected into main channel 23 from opening 22 by using a fluid pressure. The test solution injected into main channel 23 is further injected into microchannels 24. In the present embodiment, an air pressure is used as a fluid pressure. Opening 22 has a cross-section, for example, in an annular shape. Opening 22 has a diameter of, for example, 5 μm to 5 mm. In the present embodiment, one main channel 23 is connected to opening 22. One main channel 23 is arranged at a position surrounding an outer side of a plurality of microchannels 24.

Main channel 23 includes inlet-side end 23a that communicates with opening 22 and an outlet-side end 23b located opposite to inlet-side end 23a Main channel 23 that extends from opening 22 is further branched into the plurality of microchannels 24. Main channel 23 is connected to the plurality of microchannels 24 such that the test solution can flow thereto. The test solution that flows in from opening 22 flows to the plurality of branched microchannels 24 through main channel 23. Main channel 23 and microchannel 24 each have a rectangular cross section, and main channel 23 and microchannel 24 each have a width of, for example, 1 pn to 1 mm. Main channel 23 and microchannel 24, however, are different from each other in depth (height). For example, main channel 23 has a depth of 0.5 mm, whereas microchannel 24 has a smaller depth of 0.025 mm. Therefore, microchannel 24 is higher in channel resistance than main channel 23. With microchannels 24 being higher in channel resistance than main channel 23, after main channel 23 is once filled with the test solution that flows in from opening 22 as will be described later, the test solution can flow into the plurality of microchannels 24 substantially at the same time.

In the present embodiment, thirty-two microchannels 24 arranged as being aligned in a direction of an X axis are defined as one group, and two groups are arranged as being in a direction of a Y axis. In other words, microchannel device 2 includes a first group arranged on a Y axis positive direction side and a second group arranged on a Y axis negative direction side. The plurality of microchannels 24 each include a first-side end 24a that communicates with main channel 23 and a second-side end 24b located opposite to first-side end 24a.

The plurality of microchannels 24 included in the first group are each connected to main channel 23 arranged on the Y axis positive direction side of microchannel device 2. The plurality of microchannels 24 included in the first group are each connected to main channel 23 such that first-side ends 24a are located on the Y axis positive direction side and second-side ends 24b are located on the Y axis negative direction side. Therefore, the test solution branched from main channel 23 into the plurality of microchannels 24 included in the first group flows in the negative direction of the Y axis.

The plurality of microchannels 24 included in the second group are each connected to main channel 23 arranged on the Y axis negative direction side of microchannel device 2. The plurality of microchannels 24 included in the second group are each connected to main channel 23 such that first-side ends 24a are located on the Y axis negative direction side and second-side ends 24b are located on the Y axis positive direction side. Therefore, the test solution branched from main channel 23 into the plurality of microchannels 24 included in the second group flows in the positive direction of the Y axis.

After the plurality of microchannels 24 are branched from main channel 23, reservoir 25 is provided at a midpoint in each microchannel 24. Therefore, the test solution that flows in from opening 22 flows through main channel 23 and microchannels 24 to respective reservoirs 25.

An agent is arranged in reservoir 25, and reservoir 25 is connected to opening 22 through main channel 23 and microchannel 24. The test solution that flows in front opening 22 is stored in reservoir 25. In reservoir 25, the test solution reacts with the agent. The agent is, for example, an antimicrobial. The agent may be a solid or a liquid. The agent is placed in advance in reservoir 25. In other words, before the test solution flows into reservoir 25, the agent is placed in reservoir 25. In the present embodiment, the agent is applied to entire reservoir 25.

Reservoir 25 is formed in a shape of a parallelepiped. Reservoir 25 has a side having a length of, for example, 10 μm to 10 mm.

In FIG. 3, sixty-four (=32×2) reservoirs 25 are formed in plate-shaped member 20. An identical volume of the test solution is stored in sixty-four reservoirs 25. A type and an amount of the agent placed in sixty-four reservoirs 25 may be identical or different.

Microchannel 24 is arranged further between reservoir 25 and opening 26. Microchannel 24 is arranged along the direction of the Y axis. Microchannel 24 has one end connected to reservoir 25 and has the other end (second-side end 24b) connected to opening 26. The test solution that flows into reservoir 25 flows through microchannel 24 to opening 26.

Opening 26 is connected to the other end (second-side end 24b) of microchannel 24. Opening 26 has a cross-section, for example, in an annular shape. Opening 26 has a diameter of, for example, 5 μm to 5 mm.

Opening 26 is covered with a gas permeable membrane 27. Specifically, in FIG. 3, thirty-two openings 26 connected to the plurality of microchannels 24 included in the first group arranged on the Y axis positive direction side and thirty-two openings 26 connected to the plurality of microchannels 24 included in the second group arranged on the Y axis negative direction side are arranged to face each other. Therefore, sixty-four (=32×2) openings 26 are arranged along the direction of the X axis in a central portion of microchannel device 2. Sixty-four openings 26 are covered with single gas permeable membrane 27. Instead of single gas permeable membrane 27 that covers sixty-four openings 26, two gas permeable membranes 27 may selectively cover thirty-two openings 26 included in the first group and thirty-two openings 26 included in the second group. Gas permeable membrane 27 may cover at least one of sixty-four openings 26.

Gas permeable membrane 27 performs a function to allow passage of gas and not to allow passage of liquid therethrough. Examples of a material for gas permeable membrane 27 include polytetrafluoroethylene (PTFE). Gas permeable membrane 27 is preferably water-repellent. Gas permeable membrane 27 has a thickness of 1 mm or less.

Gas permeable membrane 27 is fixed to plate-shaped member 20 by adhesion by an adhesive or ultrasonic welding. Examples of the adhesive include photocurable resin, thermosetting resin, and pressure-sensitive resin.

Collection portion 40 is provided at outlet-side end 23b of main channel 23. Collection portion 40 is a portion where some of the test solution that flows into main channel 23 through opening 22 is collected.

Opening 29 is provided at an upper portion of collection portion 40. Opening 29 is covered with gas permeable membrane 27a. From opening 22 to collection portion 40, the test solution can flow through main channel 23. Elastic member 3a of opening and closing unit 30 of test apparatus 100 is pressed against gas permeable membrane 27a from above or is separated from gas permeable membrane 27a to close or open opening 29.

A channel cross-sectional area of a connection channel 44 (see FIG. 7) provided to collection portion 40 is larger than a channel cross-sectional area of microchannel 24 and a channel cross-sectional area of main channel 23. Therefore, collection portion 40 is smaller in channel resistance than microchannel 24 and main channel 23. With collection portion 40 being smaller in channel resistance than microchannel 24 and main channel 23, when air is sent from opening 22 while opening 29 is open, the test solution that remains in main channel 23 is discharged to collection portion 40.

In other words, in test apparatus 100, by closing the opening 29, the test solution that flows into main channel 23 is not discharged to collection portion 40, and by opening the opening 29, the test solution that remains in main channel 23 is discharged to and collected into collection portion 40.

Gas permeable membrane 27a may be made of a material same as or different from the material for gas permeable membrane 27 that covers openings 26, and it is sufficient for gas permeable membrane 27a to perform a function to allow passage of gas and not to allow passage of liquid therethrough. Examples of a material for gas permeable membrane 27a include polytetrafluoroethylene (PTFE). Gas permeable membrane 27a is preferably water-repellent. Gas permeable membrane 27a has a thickness of 1 mm or less. Gas permeable membrane 27a is fixed to plate-shaped member 20 by adhesion by an adhesive or ultrasonic welding. Examples of the adhesive include photocurable resin, thermosetting resin, and pressure-sensitive resin.

By providing gas permeable membrane 27a, it is possible to reduce the risk of the test solution flowing over opening 29 when the test solution is discharged to collection portion 40. In the case in which a sufficiently large size for collection portion 40 is ensured and there is a low possibility of the test solution flowing over opening 29, gas permeable membrane 27a may not be provided.

Main channel 23 also includes a sealing portion 28 between outlet-side end 23b and a connection portion 24c between main channel 23 and microchannel 24 located at a position closest to outlet-side end 23b of the plurality of microchannels 24. Sealing portion 28 is sealed after the test solution is discharged to collection portion 40. Such an operation causes main channel 23 and collection portion 40 to be physically cut off from each other to make main channel 23 and collection portion 40 independent from each other and hence, it is possible to surely prevent the test solution discharged to collection portion 40 from flowing back to main channel 23 (microchannel 24). A method of sealing the sealing portion 28 is not particularly limited. For example, sealing may be provided by pouring silicone oil, being a sealing material, or may be provided by fitting a sealing component into sealing portion 28. In the present embodiment, for example, a sealing material applied by applicator 32 is poured into sealing portion 28. In the case in which a method of sealing opening 22 or the like differs from a method of sealing the sealing portion 28, test apparatus 100 performs a function to achieve the respective sealing methods.

Main channel 23 may further include a sealing portion between inlet-side end 23a and a connection portion between main channel 23 and microchannel 24 located at a position closest to inlet-side end 23a of the plurality of microchannels 24. In the same manner as sealing portion 28, the sealing portion provided in the vicinity of inlet-side end 23a is sealed after the test solution is discharged to collection portion 40. Such an operation causes opening 22 and main channel 23 to be physically cut off from each other to make opening 22 and main channel 23 independent from each other and hence, it is possible to surely prevent the test solution in main channel 23 from flowing toward opening 22. The sealing method is not particularly limited.

[Injection Method of Test Apparatus]

FIG. 4 is a flowchart for illustrating an injection method of the test apparatus. Referring to FIG. 4, the injection method of test apparatus 100 according to the present embodiment is described. First, controller 50 of test apparatus 100 controls a motor of pipet nozzle driver 12 to move pipet nozzle 15 to a position of prescribed test solution container 5, and then controls pump 14 to suction a test solution in test solution container 5 from a tip end of pipet chip 1 (step S11). Controller 50 controls the motor of pipet nozzle driver 12 to move pipet nozzle 15 to a position of opening 22 in microchannel device 2 (step S12).

A position of opening and closing unit 30 with respect to pipet nozzle 15 is preset according to a position of opening 29 in collection portion 40 with respect to opening 22 in microchannel device 2. More specifically, the position of opening and closing unit 30 is preset such that elastic member 30a of opening and closing unit 30 is located at a position above opening 29 when pipet nozzle 15 is moved to the position of opening 22. Therefore, when pipet nozzle 15 is aligned with the position of opening 22 in microchannel device 2 in step S12, elastic member 30a of opening and closing unit 30 is moved to a position right above opening 29 in collection portion 40. Opening and closing unit 30 may be moved by a movement mechanism different from the movement mechanism (pipet nozzle driver 12) that moves pipet nozzle 15.

Controller 50 controls opening and closing driver 31 to move elastic member 30a to a position at which elastic member 30a closes opening 29 in collection portion 40 (step S13). More specifically, controller 50 causes elastic member 30a to be pressed against gas permeable membrane 27a to close entire opening 29 by elastic member 30a.

After opening 29 is closed, controller 50 controls pump 14 to discharge a test solution from the tip end of pipet chip 1, thus injecting the test solution into the channels (main channel 23, microchannels 24) of microchannel device 2 (step S14).

Controller 50 determines whether the test solution is injected into all channels of microchannel device 2 (step S15). Controller 50 determines whether the test solution is injected into all channels of microchannel device 2 based on, for example, a time period during which the test solution is injected into the channels of microchannel device 2 and an amount of the test solution remaining in pipet chip 1. When the test solution is not injected into all channels of microchannel device 2 (NO in step S15), controller 50 returns the process to step S14.

When the test solution is injected into all channels of microchannel device 2 (YES in step S15), controller 50 controls opening and closing driver 31 to move elastic member 30a from the position at which elastic member 30a closes opening 29 to open opening 29 (step S16). Controller 50 controls pump 14 to discharge air from the tip end of pipet chip 1, thus discharging the test solution that remains in main channel 23 to collection portion 40 (step S17).

Controller 50 controls pipet nozzle driver 12 and application driver 34 to pour a sealing material into sealing portion 28 (step S18). More specifically, controller 50 controls pipet nozzle driver 12 to move applicator 32 to a position right above sealing portion 28. Then, controller 50 controls application driver 34 to move applicator 32 to a position at which the sealing material can be poured into sealing portion 28. Thereafter, controller 50 drives pump 33 to pour the sealing material into sealing portion 28.

Controller 50 controls pipet nozzle driver 12 to move applicator 32 to the positions of openings 22, 26, 29, and then controls application driver 34 to apply the sealing material to openings 22, 26, 29 (step S19).

By performing the control according to the above-mentioned process, test apparatus 100 injects the test solution into microchannel device 2 and causes the test solution to be discharged to collection portion 40, and then applies the sealing material to the openings or the like formed in microchannel device 2.

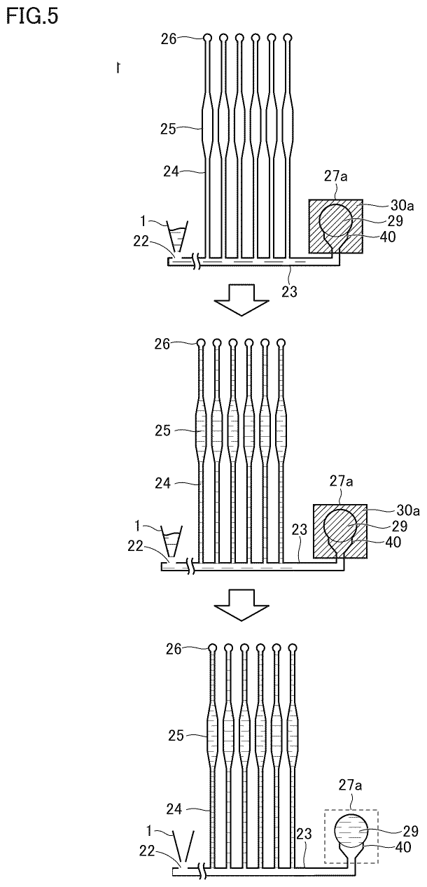

FIG. 5 is a diagram for illustrating a flow of a test solution when the test solution is injected by the test apparatus. Test apparatus 100 injects the test solution into main channel 23 from opening 22 while opening 29 is closed but openings 26 are open. Each microchannel 24 is higher in channel resistance than main channel 23. Therefore, when the test solution flows into main channel 23, as shown in an upper part of FIG. 5, first, main channel 23 is filled with the test solution. After entire main channel 23 is filled with the test solution, as shown in a middle part of FIG. 5, the test solution flows into the respective microchannels 24. At this point of operation, openings 26 are open but opening 29 is closed and hence, the test solution flows into the respective microchannels 24 and reservoirs 25 without flowing into collection portion 40.

After the respective microchannels 24 and reservoirs 25 are filled with the test solution, test apparatus 100 removes elastic member 30a of opening and closing unit 30, elastic member 30a closing opening 29, thus sending air from opening 22 while opening 29 is open. As a result, as shown in a lower part of FIG. 5, the test solution that remains in main channel 23 is discharged to collection portion 40. Air discharged from pipet chip 1 for injecting the test solution can be used as air sent from opening 22.

At this point of operation, though openings 26 are open, microchannels 24 is higher in channel resistance than collection portion 40 and hence, the test solution in main channel 23 is discharged to collection portion 40 without being pushed out to microchannels 24.

In microchannel device 2, the test solution that flows in from pipet chip 1 flows through opening 22 and main channel 23, and microchannels 24, reservoirs 25, and openings 26 are then filled with the test solution. In microchannel device 2, the plurality of microchannels 24 communicate with one another through main channel 23. Therefore, in the case in which the test solution is injected, when a height of a fluid level (fluid head) is different between respective channels or different between portions each of which is from opening 22 to each channel, the difference causes a flow of the test solution in each channel. For example, when a fluid head is different between the plurality of microchannels 24, a flow of the test solution is produced between the channels for eliminating the difference. As a result, a flow of the test solution is produced in reservoir 25 in microchannel 24 and hence, a correct result may not be observed.

In test apparatus 100 according to the present embodiment, after the test solution is injected into the channels (main channel 23, microchannels 24) of microchannel device 2, the test solution that remains in main channel 23 is discharged to collection portion 40. Such an operation can make the plurality of microchannels 24 independent from each other. As a result, even when there is a difference in height of the fluid level between the plurality of microchannels 24, it is possible to prevent a flow of the test solution that is produced due to the difference in height.

[Construction of Collection Portion]

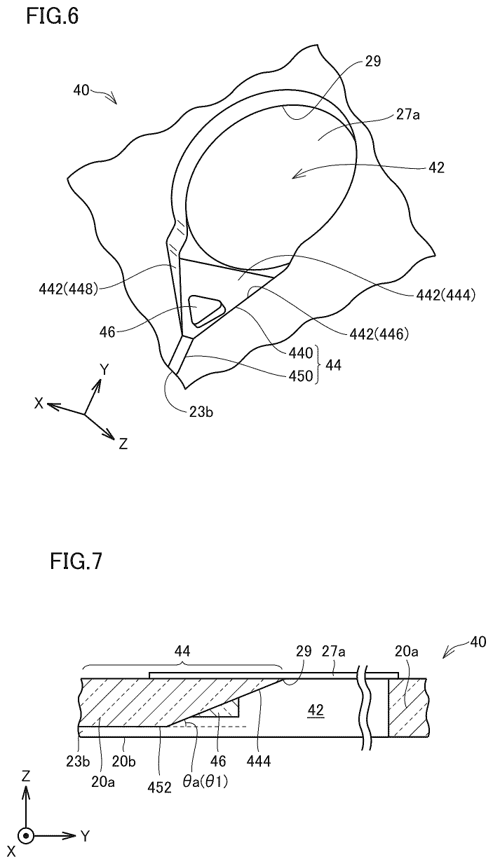

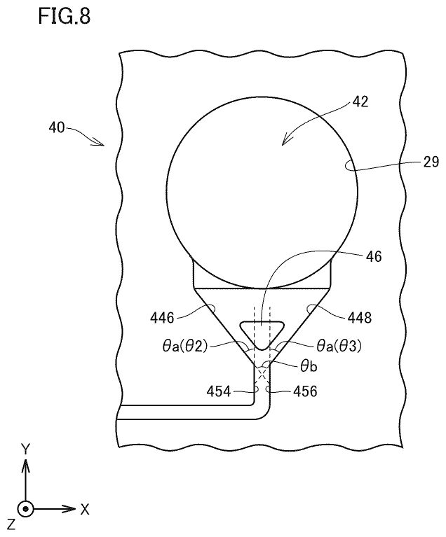

FIG. 6 is a perspective view of the collection portion. FIG. 7 is a cross-sectional view of the collection portion. FIG. 8 is a plan view of the collection portion. Referring to FIG. 6 to FIG. 8, a construction of collection portion 40 will be described. FIG. 6 is a diagram of collection portion 40 as viewed from the bottom in a state in which a bottom surface of the channel, that is, a member (a second plate-shaped member 20b in FIG. 7) that forms a surface that faces a surface provided with the opening is removed. In FIG. 8, for the sake of convenience, illustration of gas permeable membrane 27a is omitted.

Collection portion 40 includes a pool 42 that stores a test solution, connection channel 44 that connects pool 42 with outlet-side end 23b, and a protrusion 46 provided in connection channel 44.

Opening 29 is provided at an upper portion of pool 42. Opening 29 is covered with gas permeable membrane 27a. Pool 42 is a space that stores the test solution discharged from main channel 23. A volume of pool 42 is larger than a volume of entire main channel 23. Therefore, all test solution that remains in main channel 23 can be collected into pool 42. For example, in the case in which main channel 23 has a depth of 0.01 mm to 0.05 mm and a width of 0.1 mm to 1 mm, pool 42 has a diameter of 10 mm to 15 mm and a depth of 1 mm to 5 mm. For example, in the case in which main channel 23 has a depth of 0.03 mm and a width of 0.5 mm, pool 42 has a diameter of 8 mm and a depth of 2.5 mm.

Connection channel 44 includes a linear channel 450 extending from outlet-side end 23b and an inclined channel 440 that communicates with linear channel 450. Inclined channel 440 is configured to have a channel cross-sectional area increasing from linear channel 450 toward pool 42, that is, from outlet-side end 23b toward pool 42.

Inclined channel 440 has at least one inclined surface 442 that is inclined toward the outside of inclined channel 440. In the present embodiment, inclined channel 440 has three inclined surfaces 442. More specifically, inclined surfaces 442 are formed on two surfaces that form side surfaces of inclined channel 440 and on a surface of inclined channel 440 on a side where opening 29 and opening 22 are formed.

Specifically, inclined channel 440 has a first inclined surface 444, a second inclined surface 446, and a third inclined surface 448. First inclined surface 444 is formed on the surface of inclined channel 440 on the side where opening 29 and opening 22 are formed. Second inclined surface 446 and third inclined surface 448 form a tapered portion having a channel width increasing from outlet-side end 23b toward pool 42. For example, inclined channel 440 has a width of 0.1 mm to 1 mm and a depth of 0.01 mm to 0.05 mm at an end thereof close to linear channel 450, and has a width of 5 to 6 mm and a depth of 1 mm to 5 mm at an end thereof close to pool 42, and has a length, measured from the end thereof close to main channel 23 to the end thereof close to pool 42, of 2 to 6 mm.

An inclination angle θa of a channel surface extending from inclined surface 442 is less than 90 degrees. More specifically, an inclination angle θ1 of first inclined surface 444 with respect to a horizontal plane 452 is less than 90 degrees. An inclination angle θ2 of second inclined surface 446 with respect to a first side surface 454 is less than 90 degrees. An inclination angle θ3 of third inclined surface 448 with respect to a second side surface 456 is less than 90 degrees. A taper angle θb of the tapered portion formed between second inclined surface 446 and third inclined surface 448 is less than 180 degrees inclination angle θa is, for example, 15 degrees to 45 degrees. Inclination angle θ1, inclination angle θ2, and inclination angle θ3 may be identical or different.

Protrusion 46 is arranged in connection channel 44 to generate air bubbles between protrusion 46 and inner walls of connection channel 44 to close connection channel 44, upon receiving the test solution discharged from main channel 23. When air is sent from opening 22 to discharge the test solution that remains in main channel 23 to collection portion 40, the test solution and air collide with protrusion 46, so that air bubbles are generated in connection channel 44 at portions between the inner walls of connection channel 44 and protrusion 46 at a boundary portion between the test solution and the air. The channel between protrusion 46 and the inner wall of connection channel 44 has a small channel cross-sectional area and hence, connection channel 44 is closed by the air bubbles. The air bubbles are retained in connection channel 44 due to surface tension. As a result, such air bubbles can prevent the discharged test solution from flowing back to main channel 23.

In the present embodiment, protrusion 46 is provided to inclined channel 440 by using inclined surface 442 as a placement surface. More specifically, placement surface for protrusion 46 is first inclined surface 444. Protrusion 46 has a shape tapered toward outlet-side end 23b when microchannel device 2 is viewed in a plan view. Protrusion 46 has a size at which protrusion 46 does not completely close the channels of inclined channel 440.

For example, protrusion 46 has a size that allows a channel having a width substantially equal to the width of linear channel 450 or main channel 23 to be formed on both sides of protrusion 46 when microchannel device 2 is viewed in a plan view. For example, when microchannel device 2 is viewed in a plan view, protrusion 46 has a size that allows a distance between protrusion 46 and the inner wall of inclined channel 440 to be 0.1 mm to 1 mm. More specifically, in the case in which inclined channel 440 has a width of 0.5 mm and a depth of 0.03 mm at the end thereof close to main channel 23, has a width of 5.5 mm and a depth of 2.5 mm at the end thereof close to pool 42, and has a length, measured from the end thereof close to main channel 23 to the end thereof close to pool 42, of 4 mm, the size of protrusion 46 is as follows, for example. Protrusion 46 is a quadrangular pyramid with an equilateral triangular shape having a side length of 1 mm when microchannel device 2 is viewed in a plan view. When microchannel device 2 is viewed in a plan view in this case, a channel having a width of 0.5 mm is formed on either side of protrusion 46.

In addition to air bubbles of a sufficient size being generated due to the provision of protrusion 46, the channels on the periphery of protrusion 46 and linear channel 450 have sufficiently small channel cross-sectional areas and hence, air bubbles generated by protrusion 46 can close connection channel 44 and can be retained in connection channel 44 due to surface tension.

Plate-shaped member 20 includes a first plate-shaped member 20a having a plurality of openings and the channel structure, and second plate-shaped member 20b layered on first plate-shaped member 20a. A thickness of first plate-shaped member 20a and second plate-shaped member 20b is set, for example, to 0.5 mm to 3 mm, although it is not particularly limited. Though second plate-shaped member 20b is directly fixed to first plate-shaped member 20a by ultrasonic welding, it may be fixed by an adhesive.

First plate-shaped member 20a and second plate-shaped member 20b are each formed of a transparent material, and formed into a shape of a rectangular plate when microchannel device 2 is viewed from above. Examples of the material for first plate-shaped member 20a and second plate-shaped member 20b include acrylic resin such as polymethyl methacrylate resin and glass.

Openings and the channel structure are formed in first plate-shaped member 20a More specifically, in first plate-shaped member 20a, opening 22, main channel 23, microchannels 24, reservoirs 25, openings 26, and collection portion 40 provided with opening 29 are formed.

Second plate-shaped member 20b is a flat member, and functions as a bottom surface for the respective channels. More specifically, second plate-shaped member 20b functions as a lower surface for opening 22, main channel 23, microchannels 24, reservoirs 25, openings 26, and collection portion 40. Specifically, of wall surfaces that form respective channels of microchannel device 2, the surface (the surface formed by second plate-shaped member 20b) that faces the surface having opening 22 is flat.

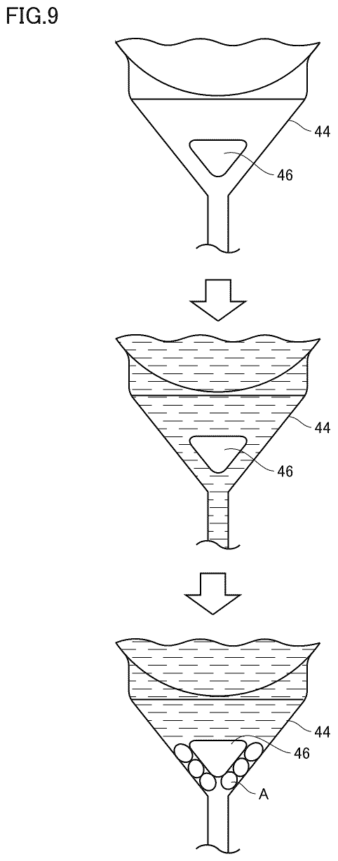

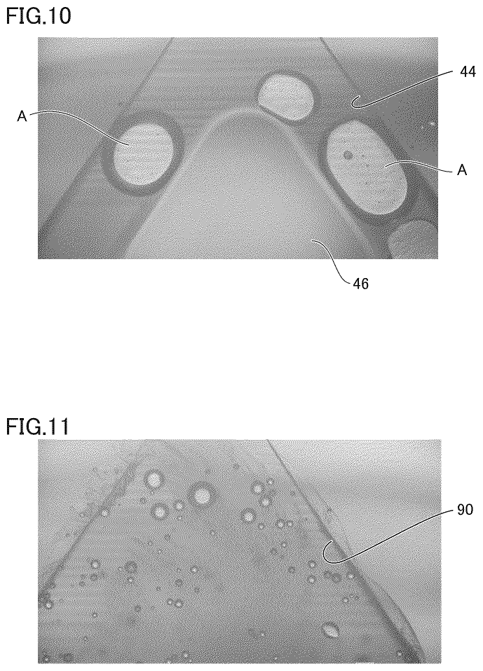

FIG. 9 is a diagram schematically showing states of the collection portion before and after the test solution is discharged. FIG. 10 is an image showing a state after the test solution is discharged to the collection portion. FIG. 11 is an image showing a state after a test solution is discharged to the collection portion in a comparison example. A connection channel 90 in the comparison example is not provided with a protrusion.

Test apparatus 100 discharges air from the tip end of pipet chip 1 to discharge the test solution that remains in main channel 23 to collection portion 40. When air pressure applied at the time of discharging the test solution is released, the test solution in connection channel 44 may flow back into main channel 23 due to a capillary action. For this reason, there is a demand for the test solution to be retained in collection portion 40.

When air is sent from opening 22 to discharge the test solution that remains in main channel 23 to pool 42, air sent from opening 22 and the test solution collide with protrusion 46, so that air bubbles A are generated at the boundary portion between the test solution and air. As shown in a lower part of FIG. 9, the generated air bubbles A have a size at which air bubbles A close connection channel 44. More specifically, as shown in FIG. 10, air bubbles A having a size large enough to close connection channel 44 are generated in connection channel 44 provided with protrusion 46. In contrast, as shown in FIG. 11, in connection channel 90 provided with no protrusion, though small air bubbles are generated, air bubbles A having a large size that closes connection channel 44 as shown in FIG. 10 are not generated. Further, as shown in FIG. 10, protrusion 46 is arranged in connection channel 44, so that the channel cross-sectional area is small. Accordingly, the channels between protrusion 46 and the inner walls of connection channel 44 are closed by the generated air bubbles.

As described above, when the test solution is discharged from main channel 23, air bubbles A that can close connection channel 44 are generated by protrusion 46. The generated air bubbles A are retained in connection channel 44 due to surface tension. Air bubbles A that are retained in connection channel 44 can prevent a backflow of the test solution from collection portion 40 to main channel 23 and, as a result, it is possible to cause the test solution to be retained in collection portion 40.

During a period in which air bubbles A are retained in connection channel 44, a backflow of the test solution can be prevented. However, when air bubbles A move from connection channel 44 to pool 42 or main channel 23, the effect cannot be obtained. In the present embodiment, after the test solution is discharged to collection portion 40, test apparatus 100 pours a sealing material to sealing portion 28 before the sealing material is applied to each of openings 26, 29, that is, at as early as possible timing. With such a configuration, it is possible to surely prevent the test solution from flowing back to main channel 23.

In order to collect the test solution in main channel 23, collection portion 40 further includes pool 42 having a volume larger than the volume of main channel 23. In the case of attempting to form pool 42 having a volume larger than the volume of main channel 23 in plate-shaped member 20, it is necessary to cause pool 42 to have a large cross-sectional area to reduce a size of microchannel device 2 and to reduce channel resistance. Therefore, connection channel 44 has a shape enlarged in the depth direction or the width direction. In the present embodiment, collection portion 40 includes inclined channel 440 having inclined surfaces 442 having inclination angle θa of less than 90 degrees and hence, the channel cross-sectional area increases from outlet-side end 23b toward pool 42.

In the case in which inclination angle θ1 of first inclined surface 444 with respect to horizontal plane 452 is 90 degrees or more, an angle formed between first inclined surface 444 and an upper surface of the channel is less than 90 degrees. Therefore, a distance between first inclined surface 444 and the upper surface of the channel is small. As a result, there may be a case in which the test solution stays at a boundary portion between first inclined surface 444 and the upper surface of the channel. In the present embodiment, inclination angle θa is less than 90 degrees. Therefore, it is possible to prevent the test solution from staying at the boundary portion between inclined surface 442 and the channel surface extending from inclined surface 442. In other words, it is possible to prevent the test solution from remaining in connection channel 44 and, as a result, a backflow of the test solution from collection portion 40 to main channel 23 can be prevented.

In the present embodiment, protrusion 46 has a shape tapered toward outlet-side end 23b. As a result, it is possible to prevent the test solution from staying at the boundary portion between protrusion 46 and the placement surface for protrusion 46. In other words, it is possible to prevent the test solution from remaining in connection channel 44 and, as a result, a backflow of the test solution from collection portion 40 to main channel 23 can be prevented.

In the present embodiment, protrusion 46 is provided in inclined channel 440 of connection channel 44 that has inclined surfaces 442. Inclined channel 440 is a channel having an enlarged channel cross-sectional area. Therefore, protrusion 46 can be easily provided.

Second plate-shaped member 20b is a flat member, and functions as a bottom surface for the respective channels. In other words, of wall surfaces that form the respective channels of microchannel device 2, the surface that faces the surface having opening 22, that is, the surface formed by second plate-shaped member 20b, is flat. Therefore, the channel structure can be simplified and hence, microchannel device 2 can be easily manufactured.

Connection channel 44 according to the present embodiment includes the tapered portion formed by second inclined surface 446 and third inclined surface 448. Taper angle θb of the tapered portion is less than 180 degrees and hence, it is possible to prevent the test solution from staying at boundary portions between the tapered portion and a channel surface extending from the tapered portion, that is, a boundary portion between second inclined surface 446 and a channel surface extending front second inclined surface 446 and a boundary portion between third inclined surface 448 and a channel surface extending from third inclined surface 448 in other words, it is possible to prevent the test solution from remaining in connection channel 44 and, as a result, a backflow of the test solution from collection portion 40 to main channel 23 can be prevented.

In the present embodiment, inclined channel 440 having first inclined surface 444 is further provided with the tapered portion and hence, it is possible to sharply increase the channel cross-sectional area. Therefore, the length of connection channel 44 can be shortened and, as a result, such a construction contributes to achieving a reduction in size of microchannel device 2.

[Modification]

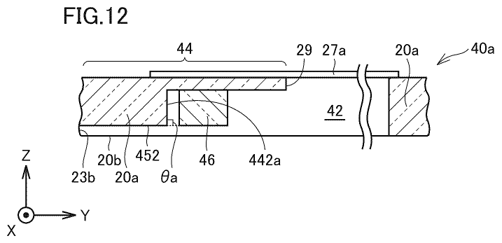

in the above-mentioned embodiment, connection channel 44 has inclined surfaces 442 having inclination angle θa of less than 90 degrees. Connection channel 44 may have an inclined surface having inclination angle θa of 90 degrees. FIG. 12 is a cross-sectional view of a collection portion according to a modification. Collection portion 40a may have an inclined surface 442a having inclination angle θa of 90 degrees. Even when inclination angle θa is 90 degrees, air bubbles are generated due to the provision of protrusion 46, so that channels between main channel 23 and pool 42 are closed by the generated air bubbles and hence, a backflow of a test solution from collection portion 40 to main channel 23 can be prevented.

In the above-mentioned embodiment, protrusion 46 is formed on inclined surface 442. It is sufficient that protrusion 46 is provided in connection channel 44 formed between pool 42 and outlet-side end 23b. For example, protrusion 46 may be provided in linear channel 450.

In the above-mentioned embodiment, inclined channel 440 includes the tapered portion formed by second inclined surface 446 and third inclined surface 448. Inclined channel 440 may not include the tapered portion.

In the above-mentioned embodiment, opening 29 is provided at the upper portion of pool 42. Opening 29 may further allow a different channel to communicate with pool 42, and may be provided in the different channel. Also in this case, opening 29 is preferably covered with gas permeable membrane 27a.

[Aspects]

The embodiment described above is understood by a person skilled in the art as specific examples of aspects below.

(Clause 1) A microchannel device according to one aspect is a microchannel device in a form of a plate used for a test in which a test solution containing a sample and an agent act on each other. The microchannel device includes an opening that receives the test solution; a main channel that communicates with the opening; a plurality of microchannels that each communicate with the main channel; and a collection portion that is provided in the main channel at an outlet-side end, the outlet-side end being located opposite to an inlet-side end communicating with the opening, the collection portion partly collecting the test solution. The collection portion includes a pool that stores the test solution discharged from the main channel, a connection channel that connects the pool with the outlet-side end, and a protrusion that is arranged in the connection channel to generate air bubbles between the protrusion and an inner wall of the connection channel to close the connection channel, upon receiving the test solution discharged from the main channel.

According to the microchannel device described in Clause 1, air bubbles generated by providing the protrusion can prevent a backflow of the test solution from the collection portion to the main channel and, as a result, it is possible to cause the test solution to be retained in the collection portion.

(Clause 2) In the microchannel device described in Clause 1, the connection channel includes an inclined channel having an inclined surface inclined toward an outside of the connection channel. An inclination angle of the inclined surface with respect to a channel surface extending from the inclined surface is less than 90 degrees.

According to the microchannel device described in Clause 2, the inclination angle is less than 90 degrees and hence, it is possible to prevent the test solution from staying at the boundary portion between the inclined surface and the channel surface extending from the inclined surface. In other words, it is possible to prevent the test solution from remaining in the connection channel and, as a result, a backflow of the test solution from the collection portion to the main channel can be prevented.

(Clause 3) In the microchannel device described in Clause 2, of wall surfaces that form channels, a surface that faces a surface provided with the opening is flat.

According to the microchannel device described in Clause 3, the channel structure can be simplified and hence, the microchannel device can be easily manufactured.

(Clause 4) According to the microchannel device described in Clause 2 or Clause 3, of wall surfaces that form the connection channel, the inclined surface is provided on a wall surface on a side where the opening is provided.

According to the microchannel device described in Clause 4, it is possible to prevent the test solution from staying at the boundary portion between the inclined surface and the channel surface extending from the inclined surface. In other words, it is possible to prevent the test solution from remaining in the connection channel and, as a result, a backflow of the test solution from the collection portion to the main channel can be prevented.

(Clause 5) According to the microchannel device described in any one of Clause 2 to Clause 4, the protrusion is provided in the inclined channel.

According to the microchannel device described in Clause 5, the inclined channel is a channel having an enlarged channel cross-sectional area and hence, the protrusion can be easily provided.

(Clause 6) In the microchannel device described in any one of Clause 1 to Clause 5, the protrusion has a shape tapered toward the outlet-side end.

According to the microchannel device described in Clause 6, it is possible to prevent the test solution from staying at the boundary portion between the protrusion and the placement surface for the protrusion. In other words, it is possible to prevent the test solution from remaining in the connection channel and, as a result, a backflow of the test solution from the collection portion to the main channel can be prevented.

(Clause 7) In the microchannel device described in any one of Clause 1 to Clause 6, the connection channel includes a tapered portion in which a channel width increases from the outlet-side end toward the pool A taper angle of the tapered portion is less than 180 degrees.

According to the microchannel device described in Clause 7, it is possible to prevent the test solution from staying at the boundary portion between the tapered portion and the channel surface extending from the tapered portion. In other words, it is possible to prevent the test solution from remaining in the connection channel and, as a result, a backflow of the test solution from the collection portion to the main channel can be prevented.

Though an embodiment of the present invention has been described, it should be understood that the embodiment disclosed herein is illustrative and non-restrictive in every respect. The scope of the present invention is defined by the terms of the claims and is intended to include any modifications within the scope and meaning equivalent to the terms of the claims.

What is claimed is:

1. A microchannel device in a form of a plate used for a test in which a test solution containing a sample and an agent act on each other, the microchannel device comprising:
an opening that receives the test solution;
a main channel that communicates with the opening;
a plurality of microchannels that each communicate with the main channel; and
a collection portion that is provided in the main channel at an outlet-side end, the outlet-side end being located opposite to an inlet-side end communicating with the opening, the collection portion partly collecting the test solution, wherein
the collection portion includes
a pool that stores the test solution discharged from the main channel,
a connection channel that connects the pool with the outlet-side end, and
a protrusion that is arranged in the connection channel to generate air bubbles between the protrusion and an inner wall of the connection channel to close the connection channel, upon receiving the test solution discharged from the main channel.

2. The microchannel device according to claim 1, wherein the connection channel includes an inclined channel having an inclined surface inclined toward an outside of the connection channel, and
an inclination angle of the inclined surface with respect to a channel surface extending from the inclined surface is less than 90 degrees

3. The microchannel device according to claim 2, wherein, of wall surfaces that form channels, a surface that faces a surface provided with the opening is flat.

4. The microchannel device according to claim 2, wherein, of wall surfaces that form the connection channel, the inclined surface is provided on a wall surface on a side where the opening is provided.

5. The microchannel device according to claim 2, wherein the protrusion is provided in the inclined channel.

6. The microchannel device according to claim 1, wherein the protrusion has a shape tapered toward the outlet-side end.

7. The microchannel device according to claim 1, wherein the connection channel includes a tapered portion in which a channel width increases from the outlet-side end toward the pool, and
a taper angle of the tapered portion is less than 180 degrees.
